(19) United States
(12) Patent Application Publication
(10) Pub. No.: US 2023/0059334 A1
(43) Pub. Date: Feb. 23, 2023

(54) NUCLEIC ACID ANALYSIS METHODS AND APPARATUS

(71) Applicant: BASF SE, Ludwigshafen (DE)

(72) Inventors: Stefan Seemayer, Ludwigshafen (DE); Holger Hartmann, Ludwigshafen (DE)

(21) Appl. No.: 17/785,762

(22) PCT Filed: Dec. 17, 2020
(86) PCT No.: PCT/EP2020/086800

§ 371 Date: Jun. 15, 2022

(30) Foreign Application Priority Data
Dec. 18, 2019 (EP) 19217297.1

(51) Int. Cl. C12Q 1/6827; C12N 15/10

(52) U.S. Cl. CPC C12Q 1/6827; C12N 15/1034; C12Q 2600/156

(57) ABSTRACT

The present invention is concerned with materials and methods for nucleic acid and/or protein analysis, including materials and methods for creating and/or analysing mutant libraries. The invention in particular relates to materials and methods for correlating a property of mutants of a target nucleic acid to the respective sequences of the mutants. The invention is particularly useful for analysing saturation mutagenesis libraries and the analysis of sensitivities to chemical or physical conditions, for example temperature stability and solvent stability, and for the analysis of production properties, for example yield or cellular localisation.

The present invention is concerned with materials and methods for nucleic acid and/or protein analysis, including materials and methods for creating and/or analysing mutant libraries. The invention in particular relates to materials and methods for correlating a property of mutants of a target nucleic acid to the respective sequences of the mutants. The invention is particularly useful for analysing saturation mutagenesis libraries and the analysis of sensitivities to chemical or physical conditions, for example temperature stability and solvent stability, and for the analysis of production properties, for example yield or cellular localisation.

### BACKGROUND OF THE INVENTION

Properties of a nucleic acid and of a protein encoded by a nucleic acid depend predominantly on the sequence of the nucleic acid. To fully understand the possible properties achievable by a selected target nucleic acid it is necessary to create mutants of said target nucleic acid and analyse those mutants. The creation of mutants has therefore been practiced for a long time in the art. For the speedy creation of a large number of mutants random mutagenesis and site-directed mutagenesis methods have been developed.

Basically there exist two ways of analysing mutants of a target nucleic acid. In a first approach, the sequence of each mutant is determined individually for all mutants in a library or a subset of the generated library determined as interesting by properties identified through characterization. This process is very time consuming, laborious and costly. In particular, such methods are unable to use the advantages of massive parallelised sequencing which would reduce the amount of work and time necessary for sequencing a large number of mutants. The advantage of such methods is that the very mutant whose sequence has been determined is still available for further analysis, for example for the analysis of further properties.

An alternative approach is described by Deng et al (J Mol. Biol. 2012, 150-167; doi:10.1016/j.jmb.2012.09.014). In such methods, first a population of mutants is created. Then the population is put under a selection pressure to enrich the share of those mutants beneficial for the respective selection pressure. Finally, for the whole population obtained after selection the nucleic acid section comprising the target nucleic acid is isolated and sequenced in parallel. The sequences thus obtained are counted; the frequency of mutated amino acids is then correlated with the selection pressure applied. It is expected that the frequency of a particular mutation is higher than average if the respective amino acid change is beneficial for overcoming their selection pressure.

The advantage of such method is that massive parallel sequencing techniques can be used to facilitate the analysis of mutation frequency patterns. However, the method necessarily implies that no single mutant clones are analysed. Instead, only a mutation frequency pattern is obtained for the whole population. If access is required to any particular mutant, such mutant has to be newly constructed. By mixing nucleic acids mutated in an unpredictable way (for example created by using degenerate primers) in parallel sequencing, for example Illumina-type sequencing or 454-type sequencing, the correlation between sequence and mutant clone is lost; the sequencer only returns sequences but is unable to separate the nucleic acids of individual mutants-for such separation individual sequencing reactions would be required, thereby losing the advantages of parallel sequencing. Furthermore the method is limited to the analysis of those properties which can be translated into a selection pressure. For many interesting properties, for example pH stability of proteins or allergenicity, no straightforward method for applying a selection pressure is available. And because it cannot normally be guaranteed that all surviving members of the population will comprise a mutation of the target nucleic acid, many of the sequences obtained by parallel sequencing will be wild type sequences and thus of little or no informative value.

Also known are methods of random mutagenesis of whole genomes to create an unspecific mutant library, for example by transposon integration as described by Vandewalle et al, Characterization of genome-wide ordered sequence-tagged Mycobacterium mutant libraries by Cartesian Pooling-Coordinate Sequencing, Nature Communications 2015, doi: 10.1038/ncomms8106, Baym et al, Rapid construction of a whole-genome transposon insertion collection for Shewanella oneidensis by Knockout Sudoku, Nature Communications 2016, doi: 10.1038/ncomms13270 or Dale et al., Comprehensive Functional Analysis of the Enterococcus faecalis Core Genome Using an Ordered, Sequence-Defined Collection of Insertional Mutations in Strain OG1RF, MSystems 2018, doi: 10.1128/mSystems.00062-18. In such approaches, transposon integrations are effected in a genome and the resulting clones are pooled according to a pooling scheme to facilitate parallel sequencing. However, these approaches cannot ensure that a sequence read is attributable to a specific clone, because several clones can comprise the same mutation. Thus, such methods are inefficient.

The invention aspires to reduce or overcome the disadvantages described above for the prior art. In particular, it is an object of the invention to provide materials and methods for analysing mutants of a target nucleic acid without sacrificing the mutants for obtaining the respective mutated nucleic acid sequence of the target nucleic acid. Further objects and in particular advantages of the present invention are described hereinafter.

### BRIEF DESCRIPTION OF THE FIGURES

FIG. 1 is a schematic outline of a method for analyzing a target nucleic acid using mutants libraries according to example 1 described herein.

FIG. 2A and FIG. 2B are a schematic outline of a method for analyzing a target nucleic acid using mutants libraries according to example 2 described herein.

### SUMMARY OF THE INVENTION

Accordingly the invention provides a method for analysing a target nucleic acid, comprising the steps of
i) providing isolated members of a set of two or more site mutagenesis libraries, wherein the members of each site mutagenesis library comprise a the target nucleic acid mutated at one or more library-specific mutation sites, and wherein the mutation sites of the two or more site mutagenesis libraries differ from each other,
ii) selecting one member of each site mutagenesis library,
iii) obtaining, for each member, a probe nucleic acid of the respectively mutated target nucleic acid comprising at least the nucleotides of the respective mutation sites and adjacent nucleotides for identification of the respective member,
iv) mixing the probe nucleic acids into one mixture, and
v) sequencing the probe nucleic acids of the mixture obtained in step iv) in parallel.

The invention also provides a site saturation mutagenesis method, comprising the steps of
i) creating, for each mutation site of a target nucleic acid, a library of mutants, wherein the mutants comprise mutations of the target nucleic acid only at the mutation site or sites specific for the respective library, and
ii) analysing the libraries by a method according to the invention.

Furthermore the invention provides a sequence analyser, comprising
a) a constraint database containing definitions of mutation sites allowed in a mutant of a target nucleic acid, and
b) a sequencer for sequencing nucleic acids in parallel, and
c) a watchdog program which, when the sequencer is used for sequencing of nucleic acids allegedly conforming to the constraint definitions, indicates erroneous sequences not conforming with the constraint definitions of the constraint database or suppresses the output of erroneous sequences.

The invention also further sequence analyser, comprising
a) a definition database of library members, wherein the library members are mutants of a target nucleic acid and the position of mutations of the target nucleic acid is specific for the respective library,
b) a sequencer for sequencing nucleic acids in parallel, and
c) a deconvolution program for identification of library members according to the library member definition based on the sequences obtained by the sequencer, optionally wherein the sequence analyser is a sequence analyser as described above.

And the invention provides a sequence analyser computer program, wherein the program is
a watchdog program for a sequence analyser as described herein, and/or
a deconvolution program for a sequence analyser as described herein and/or
a correlation program for a sequence analyser as described herein.

### DETAILED DESCRIPTION OF THE INVENTION

The invention provides a method for analysing a target nucleic acid using mutants libraries. A library according to the present invention is a mixture of microorganisms or other vessels for nucleic acids and comprise one or more mutation sites of a target nucleic acid. The vessels of the respectively mutated target nucleic acid, in particular the microorganism clones thereof, are termed “member” of the library. It is preferred but not required that the library consists or consists essentially of mutants of the target nucleic acid. Preferably, the share of non-mutated target nucleic acid members of the library is less than 20%, even more preferably less than 10% of the total library. The library can be implemented as a mixture of clones in a microbial host strain; the host strain is not limited to a particular type of microorganism but can be chosen according to the skilled person's needs. Preferably the microbial host strain is selected from a microorganism of any of the genera Escherichia, Bacillus, Corynebacterium, Lactobacillus, Lactococcus, Saccharomyces, Pichia or Yarrowia.

The target nucleic acid can be any nucleic acid as long as the host strain is capable of surviving a mutation of the target nucleic acid, for example by supplementation of a growth medium with media components that are required due to a possible mutation of the target nucleic acid. For example, where the target nucleic acid codes for an enzyme critical in the microbial hosts metabolism, the host strain should be maintained in a medium supplementing the respective substance that may no longer be synthesised by the microbial host's metabolism on its own.

In particular, the target nucleic acid can be a transcription effector nucleic acid, for example a promoter, terminator, inhibitory nucleic acid (for example RNAi), guiding nucleic acid (for example single guide RNA or crRNA and tracer RNA for CRISPR applications) or a transcription factor binding site. The target nucleic acid can also be a tRNA or rRNA. It is particularly preferred that the target nucleic acid codes for a protein. The protein can be of any type or length and can be for example and enzyme.

Each mutation site of a library can be one or more nucleotides, preferably one or more adjacent nucleotides. Particularly preferred mutation site definitions are described hereinafter.

The method for analysing a target nucleic acid according to the present invention comprises the step of providing isolated members of a set of two or more site mutagenesis libraries, wherein the members of each site mutagenesis library comprise a target nucleic acid mutated at one or more mutation sites, and wherein the mutation sites of the two or more site mutagenesis libraries differ from each other. The target nucleic acid is thus mutated according to a predefined pattern of mutation sites such that the mutation sites are specific for each library of mutants of the target nucleic acid, that is, each library comprises mutations at known positions. For example, where the target nucleic acid codes for a protein, one library may comprise mutants of the target nucleic acid mutated only at position 1 of the peptide sequence and another library contains mutants mutated only at position 2 of the amino acid sequence of the protein coded for by the target nucleic acid. As will be seen below, it is preferred but not necessary that the mutation sites of the respective libraries do not overlap as long as the mutation sites are specific for each library. By providing isolated members of a set of two or more site mutagenesis libraries as defined according to the present invention it is possible to attribute every mutant sequence of the target nucleic acid to exactly one member of one library. This way the invention allows to simultaneously use the advantages of individual sequencing, that is, the sequenced clones can be maintained as such in isolated form, and the advantages of massive parallel sequencing, in particular the speed of obtaining many sequences without having to prepare many individual sequencing reactions which would be time-consuming and liable to errors.

The method for analysing a target nucleic acid using mutants libraries further comprises the step of selecting one member of each site mutagenesis library. A typical way of selecting library members in the art is to plate out the microbial hosts making up a library and to pick one of the resulting colonies. By selecting only one member per library the invention avoids reading the same sequence multiple times: if the target nucleic acid region of all members of a library would be sequenced in parallel, then many clones would be detected having the identical mutation because each library is characterised by a limited number of possibly mutated nucleotides. Furthermore, as described above for the population analysis approach it would not be possible to maintain a correspondence between a particular member of the library and a particular sequence of the mutated target nucleic acid. The method according to the present invention ensures that the picked colony contains the same genetic information. Such a picked colony from one generated library can be mixed with one or more colonies from other generated libraries of non-overlapping mutations while still allowing the identity of the mutation to be recovered

The method for analysing a target nucleic acid using mutants libraries according to the invention further comprises the step of obtaining, for each member, a probe nucleic acid of the respectively mutated target nucleic acid comprising at least the nucleotides of the respective mutation sites and adjacent nucleotides for identification of the respective member. This way, sequencing of the nucleic acid of the selected member can be limited to interesting sections of high information content. Particularly where the great length of the parallel sequencing method is limited, the size of the probe nucleic acid can be adjusted accordingly. The probe nucleic acid can thus comprise or consist of the whole length of the target nucleic acid. In this case the probe nucleic acid necessarily comprises the nucleotides of the library specific mutation site or sites and all adjacent nucleotides. It is thus possible to identify the selected member by the sequence of the probe nucleic acid because only one of the selected members can differ from the target nucleic acid sequence at the mutation sites characterising the library of the selected member, and because only one member per library is selected, identification of the correct library essentially is the same as identification of the selected member of the library. The probe nucleic acid can also be shorter than the length of the target nucleic acid as long as the library specific mutation sites and adjacent nucleotides are present in the probe nucleic acid to allow identification of the respective member. For example where a protein comprises repetitions of a short amino acid sequence, and the mutation site or sites is adjacent to or comprised within one of the repeats, then the probe nucleic acid must comprise additional adjacent nucleotides to disambiguate the respective repeats of the amino acid sequence motive. Preferably each probe nucleic acid has a length of at least 15 nucleotides, even more preferably at least 30 nucleotides, even more preferably at least 40 nucleotides, even more preferably at least 50 nucleotides, even more preferably at least 60 nucleotides, even more preferably at least 75 nucleotides, even more preferably at least 90 nucleotides, even more preferably at least 120 nucleotides, and/or comprises the full length of the target sequence.

The method for analysing a target nucleic acid using mutants libraries according to the invention further comprises the step of mixing the probe nucleic acids into one mixture. This way the method according to the present invention avoids the need for individual sequencing of the probe nucleic acids and can instead use the advantages conveyed by parallel sequencing methods.

In a further step the method for analysing a target nucleic acid using mutants libraries according to the present invention comprises the sequencing of all probe nucleic acids of the mixture obtained in step iv) in parallel. The method according to the present invention is not limited to a particular type of parallel sequencing. Preferably, the parallel sequencing is of a next generation type sequencing technology, for example Illumina-type sequencing by synthesis, 454 type pyrosequencing, Nanopore sequencing or SMRT sequencing. It is a particular advantage of the present invention that the method described herein allows to use parallel sequencing methods which are normally agnostic to the members of a library used to construct the mixture that is sequenced while at the same time maintaining a one-to-one correspondence between every sequence obtained by parallel sequencing and the respective members of all libraries selected in step ii). Thus the method of the present invention allows to use particularly fast and reliable sequencing methods which typically have a low cost per sequenced nucleotide. And, as described above, the method of the present invention does not only turn out sequences but does also leave the library members themselves to the disposition of the skilled person. In the example described above, the skilled person could again pick a selected colony of a site mutagenesis library, multiply the microbial hosts thus obtained and subject them to further analysis.

A further advantage of the method of the present invention is that essentially any method for creating mutants can be applied as long as the mutations induced are confined to the mutation sites of the respective libraries. An example of such site specific mutagenesis techniques is described in WO201926211A1 and WO2009152336A1.

Furthermore, the method of the present invention advantageously allows to perform sequencing error detection during or after sequencing of the probe nucleic acids. Due to the definition of the libraries, that is, mutations are limited to library specific sites, the sequences obtained in step v) can deviate from the sequence of the target nucleic acid only in a very constrained way. For example, when a sequence is detected comprising mutations at 2 sites which are not both provided according to the mutation site definitions of a single library, then this sequence must be erroneous. It is thus also possible to correct sequences if the correct sequence is doubtful at a particular position. Because only certain nucleotides are allowable in each probe nucleic acid for each position, it is possible to select with without ambiguity the correct alternative in those cases where the sequencing reaction renders two or more alternative readings at a particular position.

In the method for analysing a target nucleic acid using mutants libraries according to the present invention, preferably steps ii) and iii) are repeated to select at least one additional member of one or more site mutagenesis libraries and, for each round of repetition of steps ii) and iii), the probe nucleic acids are marked by a round specific nucleic acid tag before step iv). It is a particular advantage that the method of the present invention does not rely on tagging each member of each library whose sequence is to be determined. Instead, in those cases where more than one member per library is to be analysed, then only tags have to be applied to the probe nucleic acids according to the total number of members selected per library. Preferably, the skilled person will first select one member of each site mutagenesis library and obtain the corresponding probe nucleic acid thereof, then these probe nucleic acids are combined to form a first mixture. To all probe nucleic acids of this first mixture an identical nucleic acid tag is then attached. In a next round, the skilled person again selects one member per library, obtains the respective probe nucleic acids and combines them into a second mixture. To all probe nucleic acids of the second mixture a tag nucleic acid different from that applied in the first round is then attached. The repetitions of members selection, obtaining of probe nucleic acid and attachment of a round specific tags can then be repeated as often as required. The tagged mixtures of the respective rounds of repetition are then combined into one mixture as described in step iv) of the method according to the present invention. Optionally no tag is attached in the first round, that is, only for the probe nucleic acids of the second and further members selected from a library a nucleic acid tag is attached.

The present invention thus allows, for one additional reaction per repetition, to increase the number of members analysed for each library. It is a particular advantage that the method of the present invention thus facilitates the simultaneous sequencing of a large number of mutants of a target nucleic acid without compromising the one-to-one relation between selected members and sequences.

The concentration of probe nucleic acids in the mixture or mixtures for sequencing is preferably chosen high enough to achieve oversampling, i.e. more than one copy of each probe nucleic acid (which can optionally comprise a tag as described above) is read during sequencing step v). Preferably, each probe nucleic acid is sequenced at least 3 times in step v) (3-fold oversampling), even more preferably at least 5 times (5-fold oversampling), even more preferably at least 10 times (10-fold oversampling).

Preferably a total of up to 100000 probe nucleic acids, optionally comprising tags as described above, are sequenced in parallel in step v), more preferably at least 5 different nucleic acids, even more preferably 5 to 70000 nucleic acids, even more preferably at least 25 different nucleic acids, even more preferably 25 to 65000 nucleic acids, even more preferably at least 50 different nucleic acids, even more preferably 50 to 10000 nucleic acids. The number of nucleic acids sequenced in parallel is chosen according to the abilities of the sequencing method employed in step v), the required liquid volumes for each sample and capacity of screening plates.

In the method for analysing a target nucleic acid using mutants libraries of the present invention, preferably for at least one member selected in step ii) a property dependent on the target nucleic acid is determined, and preferably the same property is determined for each selected member. The property can be any property other than the mutated sequence of the target nucleic acid, because it would be pointless to re-sequence a nucleic acid whose sequence is already known. It is a particular advantage of the method of the present invention that any measurable property can be analysed for the library members as long as the property does not prevent the creation of the library. For example, mutants of a nucleic acid constitutive for survival of the host strain can be created and analysed if the host strain is grown in a medium which complements for any loss of function caused by mutations of the essential target nucleic acid. In particular, the method of the present invention is not limited to the analysis of mutation effects on the survival of a host strain under selection conditions. Thus, preferably the property analysed is not antibiotic resistance.

Preferably the property is a property of the target nucleic acid as such or, even more preferably, of a protein encoded by the target nucleic acid. Properties of the target nucleic acid as such typically require that the target nucleic acid is functionally connected to a reporter gene. For example where the target nucleic acid is a promoter, then the target nucleic acid can be fused to a reporter gene to facilitate expression strength of the promoter under the chosen cultivation conditions. Furthermore two or more properties may be analysed in parallel in the same or different analytical reactions.

Preferably the property is selected from one or more of
expression,
secondary, tertiary or quarternary structure, folding efficiency, aggregation, multimerization
temperature stability, pH stability, solvent stability, detergent stability, protease stability, binding stability, solubility, protease activity, storage stability, storage stability in detergent, residual activity after storage, stability against proteolytic degradation,
changes of kinetic parameters, enzymatic activity at low temperatures, preferably at temperatures at or below 30° C., in particular below 20° C.,
change of substrate, substrate specificity, enantioselectivity, cofactor dependence, inhibitor specificity, inhibition kinetics,
immunogenicity, toxicity, allergenicity, wash performance,
location in the cytoplasm, spore, cell membrane, cell organelle lumen or membrane, cell wall, excretion.

The list of preferred properties described above is testimony of the versatility of the method for analysing a target nucleic acid using mutants libraries according to the present invention. The method is thus essentially applicable to for example any mutational analysis of any protein's function in a host cell.

Particularly preferred properties are described hereinafter by giving appropriate definitions:

“Enzyme properties” include, but are not limited to catalytic activity as such, substrate/cofactor specificity, product specificity, increased stability during the course of time, thermostability, pH stability, chemical stability, and improved stability under storage conditions.

The term “substrate specificity” reflects the range of substrates that can be catalytically converted by an enzyme.

“Enzymatic activity” means at least one catalytic effect exerted by an enzyme. In one embodiment, enzymatic activity is expressed as units per milligram of enzyme (specific activity) or molecules of substrate transformed per minute per molecule of enzyme (molecular activity). Enzymatic activity can be specified by the enzymes actual function, e.g. proteases exerting proteolytic activity by catalyzing hydrolytic cleavage of peptide bonds, lipases exerting lipolytic activity by hydrolytic cleavage of ester bonds, etc.

Enzymatic activity may change during storage or operational use of the enzyme. The term “enzyme stability” relates to the retention of enzymatic activity as a function of time during storage or operation.

To determine and quantify changes in catalytic activity of enzymes stored or used under certain conditions over time, the “initial enzymatic activity” is measured under defined conditions at time cero (100%) and at a certain point in time later (x%). By comparison of the values measured, a potential loss of enzymatic activity can be determined in its extent. The extent of enzymatic activity loss determines an enzymes stability or non-stability.

Parameters influencing the enzymatic activity of an enzyme and/or storage stability and/or operational stability are for example pH, temperature, and presence of oxidative substances:

“pH stability”, which refers to the ability of a protein to function at a particular pH. In general, most enzymes are working under conditions with rather high or rather low pHs. A substantial change in pH stability is evidenced by at least about 5% or greater modification (increase or decrease) in the half-life of the enzymatic activity, as compared to the enzymatic activity at the enzyme's optimum pH.

The terms “thermal stability” and “thermostability” refer to the ability of a protein to function at a particular temperature. In general, most enzymes have a finite range of temperatures at which they function. In addition to enzymes that work in mid-range temperatures (e.g., room temperature), there are enzymes that are capable of working in very high or very low temperatures. Thermostability may be characterized by what is known as the T50 value (also called half-life, see above). The T50 indicates the temperature at which 50% residual activity is still present after thermal inactivation for a certain time, compared with a reference sample which has not undergone thermal treatment. A substantial change in thermal stability is evidenced by at least about 5% or greater modification (increase or decrease) in the half-life of the enzymatic activity when exposed to given temperature.

The terms “thermal tolerance” and “thermotolerance” refer to the ability of a protein to function after exposure to a particular temperature, such as a very high or very low temperature. A thermotolerant protein may not function at the exposure temperature, but will function once returned to a favorable temperature.

“Oxidative stability”, which refers to the ability of a protein to function under oxidative conditions, in particular in the presence of various concentrations of H2O2, peracids and other oxidants. A substantial change in oxidative stability is evidenced by at least about a 5% or greater modification (increase or decrease) in the half-life of the enzymatic activity, as compared to the enzymatic activity present in the absence of oxidative compounds.

“Stability to proteolysis” refers to the ability of a protein to withstand proteolysis. Proteolysis is the breakdown of proteins (e.g. enzymes) into peptides or amino acids. Enzymatically, proteolysis is catalyzed by proteases, enzymes which have proteolytic activity. Non-enzymatically induced proteolysis can be caused by extremes of pH and/or high temperatures. Stability to proteolysis herein is distinguished from stabilization of proteases.

Enzymes storage stability normally is impaired in aqueous solution in the course of time. This can be avoided by storage of enzymes under non-hydrous conditions. Where non-hydrous conditions are not applicable, e.g. in compositions naturally comprising water, different or additional strategies need to be applied. Stabilization of proteolytic enzymes (proteases) by inhibition is a common technique to prevent proteolytic degradation (proteolysis) of proteins (such as enzymes) into peptides or amino acids (which may inactivate the functionality of e.g. an enzyme). Stabilization of proteases commonly makes use of reversible inhibition of the enzyme.

“Half-life of enzymatic activity” is a measure for time required for the decaying of enzymatic activity to fall to one half (50%) of its initial value.

“Enzyme inhibitors” slow down the enzymatic activity by several mechanisms as outlined below. Inhibitor binding is either reversible or irreversible. Irreversible inhibitors usually bind covalently to an enzyme by modifying the key amino acids necessary for enzymatic activity. Reversible inhibitors usually bind non-covalently (hydrogen bonds, hydrophobic interactions, ionic bonds). Four general kinds of reversible inhibitors are known:

(1) substrate and inhibitor compete for access to the enzymes active site (competitive inhibition),
(2) inhibitor binds to substrate-enzyme complex (uncompetitive inhibition),
(3) binding of inhibitor reduces enzymatic activity but does not affect binding of substrate (non-competitive inhibition),
(4) inhibitor can bind to enzyme at the same time as substrate (mixed inhibition).
By using enzyme inhibitors, especially reversible inhibitors, an enzyme is assumed to be stabilized. A stabilized enzyme can result from temporarily inhibiting an enzyme in its catalytic activity when compared to the catalytic activity of the same, non-inhibited enzyme. Preferably, a protease is inhibited in its proteolytic activity. Due to inhibition of proteolytic activity of at least one protease, another enzyme and the protease itself may be stabilized as their proteolytic degradation may be prevented resulting in retention of the catalytic activity of the other enzyme.

There are many methods known to one of skill in the art for immobilizing enzymes or fragments thereof, or nucleic acids, onto a solid support. Some examples of such methods include, e.g., electrostatic droplet generation, electrochemical means, via adsorption, via covalent binding, via cross-linking, via a chemical reaction or process, via encapsulation, via entrapment, via calcium alginate, or via poly (2-hydroxyethyl methacrylate). Like methods are described in Methods in Enzymology, Immobilized Enzymes and Cells, Part C. 1987. Academic Press. Edited by S. P. Colowick and N. O. Kaplan. Volume 136; and Immobilization of Enzymes and Cells. 1997. Humana Press. Edited by G. F. Bickerstaff. Series: Methods in Biotechnology, Edited by J. M. Walker.

Nucleic acids, enzymes, or consortiums or cocktails of nucleic acids or enzymes, can be immobilized by attachment to a solid support. The solid support can be placed into and removed from a process vat or other container, where the nucleic acids or enzymes are used repeatedly. In one embodiment, the solid support is selected from the group of a gel, a resin, a polymer, a ceramic, a glass, a microelectrode, and/or any combination thereof.

According to the method of the present invention, preferably each member of the libraries comprises only one mutation site or, less preferably, two or more non-adjacent mutation sites. According to the present invention a mutation site consists of one or more adjacent nucleotides. The definition of mutation sites can be different for each library such that the members of one library only comprise one mutation site and the members of another library comprise two or more nonadjacent mutation sites. If the number of mutation sites is limited to 1 per library, then this facilitates a systematic analysis of single point mutations of the target nucleic acid. This is particularly useful to create complete transcription factor binding matrices of a transcription factor binding site of the target nucleic acid or for analysing the effects of point mutations to the function of a protein. The skilled person may instead decide that each library comprises 2 or more nonadjacent mutation sites. This is for example useful to analyse the effects of multiple simultaneous amino acid substitutions to a protein encoded by the target nucleic acid, for example when modifying the active centre of an enzyme. For example when the mutation sites have the length of one codon and are separated by one codon, the mutated amino acids would typically rest on the same side of a beta sheet structure. And where the mutation sites have the length of one codon and are separated by 2 or 3 codons, then the amino acids affected by mutations will normally find themselves facing into the same direction in an alpha helix structure. The skilled person will therefore adapt the definition of mutation sites according to his needs.

Preferably each mutation site is of 1 to 9 nucleotides length, even more preferably of 2-9 nucleotides length, even more preferably of 2-6 nucleotides length, even more preferably of 3-9 nucleotides length, even more preferably of 3-6 nucleotides length, even more preferably of 3-4 nucleotides length, even more preferably of 3 nucelotides length.

Further preferably, the mutation sites of two or more libraries at most partially overlap or, even more preferably, do not overlap; most preferably the mutation sites of all libraries do not overlap. The method of the present invention requires that the mutation sites allow for the identification of a library by a sequence obtained in step v), optionally with the help of a nucleic acid tag as described above. If the mutation sites of all libraries would be identical, then in effect only one library would have been provided in step i).

It is particularly preferred that the mutation sites of all libraries do not overlap. This way, any given nucleotide position in the sequence of the target nucleic acid can be mutated in only one library. Thus, the detection of sequencing errors is particularly facilitated, because the detection of any mutation in a sequence of a probe nucleic acid immediately identifies the corresponding library; if any further mutation outside of the mutation site or sites is detected during sequencing, then this sequence is erroneous. Also as described above, if it is doubtful which of two nucleotide readings is the correct one, then the correct one may be chosen in view of all other mutations and allowable mutations according to the definition of the respective library.

In the method according to the present invention, the nucleotides of the mutation sites of one, two or more libraries preferably (a) differ from the target nucleic acid sequence by one or more arbitrary nucleotides and/or (b) differ from the target nucleic acid sequence by one or more degenerate nucleotides independently selected from [AT], [CG], [AC], [GT], [AG], [CT], [CGT], [AGT], [ACT], or [ACG], or (c) are selected from a predefined set of nucleotide or nucleotide sequences, preferably wherein the mutation site comprises or consists of a codon replaced by another codon selected from a predefined set of codons. Within the context of the present invention, the notation of upper case letters in angle brackets indicates that the respective nucleotides can be exchanged for one another. Thus for example “[AT]” means “A or T”.

As described above, the introduction of random mutations at defined positions of a target nucleic acid sequence has been well documented in the art and can be performed without any undue burden on the skilled person. The method according to the present invention thus can be advantageously applied using routine techniques and materials.

It is preferred that the nucleotides of the mutation sites of one, two or more, preferably all, libraries differ from the target nucleic acid sequence in at least one position by one or more degenerate nucleotides as described above. The use of degenerate nucleotides is well established in the prior art, see for example the international patent application publications indicated above. By using such partially degenerated nucleotides, the method according to the present invention allows for the detection of sequencing errors or errors in the synthesis of the mutant libraries. Each of the partially degenerated nucleotides excludes one or more nucleotides at the respective positions. For example where a mutation site is defined by the sequence NN[AT], any occurrence of A or T at the last position of the mutation site must be the result of a sequencing or synthesis error. Furthermore, where a mutation site is defined by NN[CG], any unwanted random introduction of a stop codon would be limited to the amber stop codon.

Even more preferably, the nucleotides of the mutation sites are selected from a predefined set of nucleotides or nucleotide sequences as described for example in US20110165627A1. The method of the present invention preferably allows for a definition of the mutation site such that the mutation site comprises or consists of a codon replaced by another codon selected from a predefined set of codons. Such method is not dependent on the use of degenerated primers and is not liable to randomly introducing unwanted stop codons. Instead, each amino acid at a given position in a sequence of a protein encoded by the target nucleic acid can be replaced by every other amino acid (and, if so wanted, by a stop codon) by simple synthesis of the target nucleic acid without the need of recourse to degenerated nucleotides. Furthermore, because each amino acid at every mutation site for all libraries is encoded by only one codon, it is possible to detect sequencing or synthesis errors whenever, at a mutation site, a codon is detected that is not comprised in the predefined set of codons. The codons of the predefined set of codons can be defined in any way. Preferably, the set of codons comprises or consists of the most preferred codon for every amino acid according to the microbial host's codon preference. The codons may also be chosen to optimise the Levenshtein distance.

The method of the present invention can be implemented in sequence analyser machines or sequence analyser computer programs. Within the present invention, a computer program can be a single monolithic computer program or may comprise modules which may be executed simultaneously or in sequence.

The present invention therefore provides a sequence analyser comprising a constraint database containing definitions of mutation sites allowed in a mutant of a target nucleic acid. As described above, the mutation sites each consist of one or more adjacent nucleotides, and the definition of mutation sites allows to differentiate between different libraries. Thus, the constraint database of the sequence analyser of the present invention reflects the definition of libraries of the method of the present invention.

The sequence analyser of the present invention also comprises a sequencer for sequencing nucleic acids in parallel. Preferred sequencing methods are described above in connection with the method for analysing a target nucleic acid using mutants libraries according to the present invention; the skilled person can choose a sequencer according to any parallel sequencing method.

Where the sequence analyser according to the present invention comprises a constraint database as described above, it also comprises a watchdog program which, when the sequencer is used for sequencing of nucleic acids allegedly conforming to the constraint definitions in the constraint database, indicates erroneous sequences not conforming with the constraint definitions of the constraint database or suppresses the output of such erroneous sequences. This way, the sequence analyser advantageously detects erroneous sequences and may either indicate to an operator that a particular member of a particular library needs additional sequencing or may refuse to provide a sequence for this member. In particular, the watchdog program may detect unexpected mutations outside of the respective mutation sites of the libraries by counting the frequency of every amino acid at every position of the sequences. As described above, the probe nucleic acids can comprise mutations only at mutation sites, thus for any given position in the probe nucleic acids the and mutated nucleotide will be the most frequent one. If a nucleotide at a given position is not the most frequent one at this position, then the nucleotide must be part of a mutation site or indicates a synthesis of sequencing error.

The invention also provides a sequence analyser comprising a definition database of library members, wherein the library members are mutants of a target nucleic acid and the position of mutations of the target nucleic acid is specific for the respective library. In this case, the sequence analyser also comprises a deconvolution program for identification of library members according to the library member definition is based on the sequences obtained by the sequencer. As described above, the method of the present invention preserves a one-to-one correlation between library members and sequences obtained during a parallel sequencing step. Thus, the sequence analyser according to the present invention allows to implement and make use of some or all of the advantages conferred by the method for analysing a target nucleic acid using mutants libraries according to the present invention.

The sequence analyser of the present invention preferably further comprises a database comprising, for at least one library member, a quality of a property dependent on the target nucleic acid, and a correlation program for creating a pairwise assignment of library member sequence to property quality of the respective library member. Thus, the sequence analyser allows to link, for example in one document, the intensity measured for a property or, for example where the property is measured according to a cardinal or classification definition, any other quality of the property to the respective library member sequence and thus to the respective library member.

The invention also provides a sequence analyser computer program, wherein the program is a watchdog program for a sequence analyser as described above, and/or a deconvolution program for a sequence analyser as described above and/or a correlation program for a sequence analyser as described above.

The invention is hereinafter further described in the accompanying examples and figures; both are not intended to limit the scope of the claims.

### EXAMPLES

### Example 1: Single-Pick Libraries

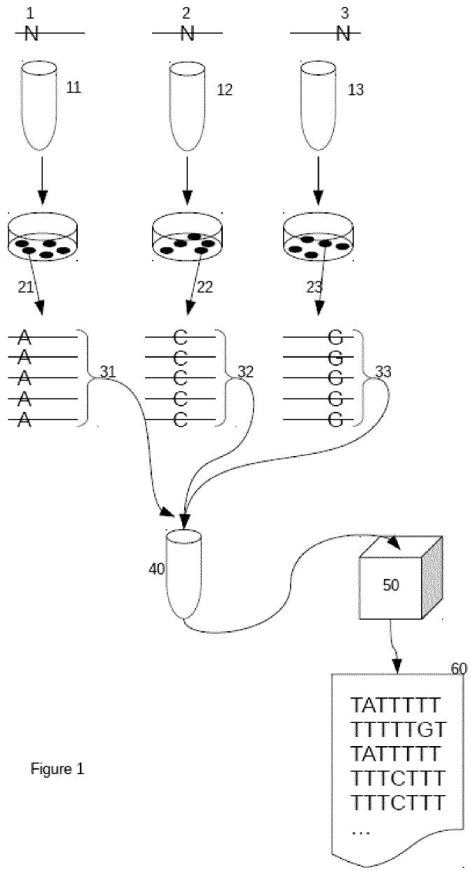

FIG. 1 is an exemplary schematic drawing and features several features which are individually and in combination with other features preferred according to the present invention. According to this example, there is first constructed a set of mutants libraries (11, 12, 13) by mutating, respectively, a target nucleic acid at specific mutation sites (1, 2, 3). In the figure the mutation sites are of single nucleotide length, the respective mutations are created by replacement of the respective wild type nucleotide with a degenerate [ACGT] nucleotide. As described above the invention is not limited to such single nucleotide replacements; the mutation sites could as well have consisted of replacements of for example three consecutive nucleotides at library specific mutation sites to effect, for each library, a codon replacement and thereby an amino acid replacement in a protein coded by the target nucleic acid.

The mutants libraries (11, 12, 13), which consist of microbial host cells comprising the respectively mutated target nucleic acid, are the plated on suitable media plates; from each plated library one library member colony (21, 22, 23) is picked. The picked colony is analysed for a property dependent on the target nucleic acid (not shown), for example enzyme activity where the target nucleic acid codes for an enzyme. If the quality of the property conforms to the skilled person's interests, for example because the enzymatic activity is significantly higher or significantly lower than the wild type enzymatic activity, then a probe nucleic acid (31, 32, 33) is created from the respective selected member.

The probe nucleic acid can be created for example by PCR amplification of the mutated target nucleic acid. As described above it is not necessary to determine the property before selecting the respective members for probe nucleic acid generation; the property can also be determined later.

The amount of probe nucleic acids (31, 32, 33) is adjusted such as to ensure that during sequencing multiple probe nucleic acids (31, 32, 33) are sequenced; in the case depicted by FIG. 1 a 5-fold oversampling is aspired.

The adjusted probe nucleic acids (31, 32, 33) are then combined into a sequencing mixture (40) and sequenced in a parallel sequencer (50). The sequencer produces a list of sequences (60).

For the purposes of this example, the wild type target nucleic acid consists of 7 consecutive T nucleotides. This way the position of mutated nucleotides in the sequences (60) obtained from the sequencer (50) are readily apparent. The sequences can be deconvoluted according to the respective mutation sites. For example, the first sequence comprises a nucleotide other than T at the second position. The first library 11 consists of mutants at this position. Thus, the first sequence must belong to the mutated target nucleic acid of member 21. The second sequence comprises a nucleotide other than T at position 6. The third library 13 consists of mutants at this position. Thus, the second sequence belongs to the mutated target nucleic of member 23 of the third library. The third nucleic acid is the same as the first nucleic acid; this is a result of the probe nucleic acid concentration adjustments (31, 32, 33) resulting in oversampling. The next two sequences are again identical due to oversampling and comprise a nucleotide other than T at the fourth position. The second library 12 consists of mutants at this position. Thus, the last two sequences shown in FIG. 1 belong to the mutated target nucleic acid of member 22 of the second library.

### Example 2: Combined Libraries and Multiple Rounds of Selection and Probe Nucleic Acid Generation

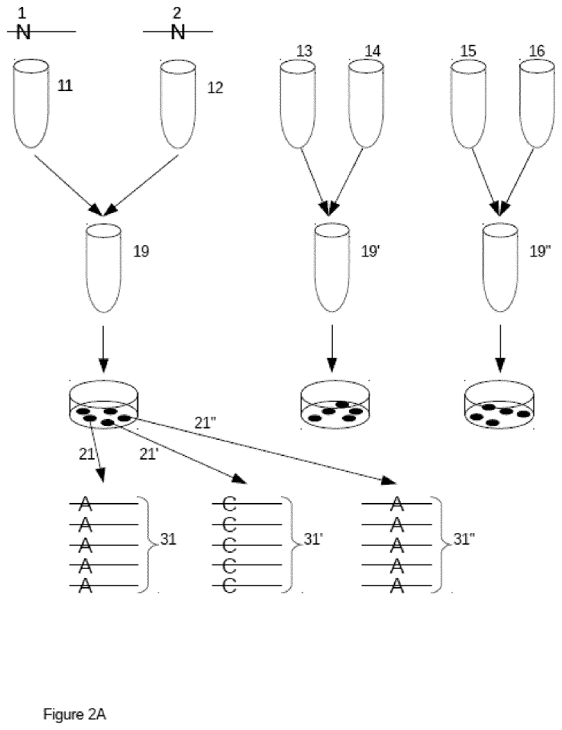

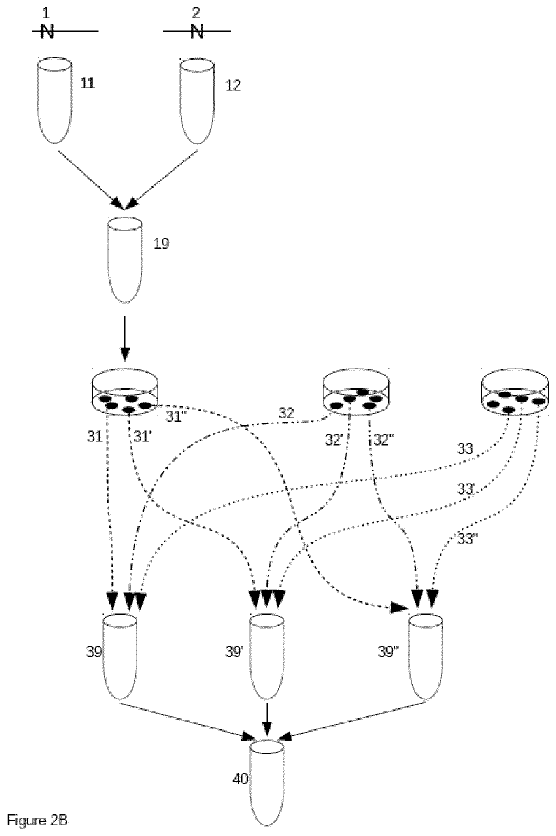

According to FIG. 2A, a target nucleic acid is mutated like in example 1 at multiple library-specific single nucleotide positions; only two such mutated target nucleic acids are shown (1, 2). The mutated target nucleic acids are comprised in respective mutants libraries (11, 12, 13, 14, 15, 16).

Two of the mutants libraries are then mixed to create combined libraries (19, 19′, 19″). The combined libraries consist of mutant libraries such that the mutation sites of the parent libraries differ from one another, preferably the mutation sites of the parent libraries do not overlap.

As in example 1, the combined mutants libraries (19, 19′, 19″), are the plated on suitable media plates; the plating is shown in FIG. 2A only for one combined library (19). From the plated combined libraries (19) individual clones (21, 21′, 21″) are picked before, after or without determining a selected property dependent on the target nucleic acid (see example 1 mutatis mutandis). As shown in FIG. 2A, a probe nucleic acid (31, 31′, 31″) is created from each of the picked combined library members (21, 21′, 21″) respectively. Because the combined library (19) consists of members of two parent libraries (11, 12), the resulting probe nucleic acids (31, 31′, 31″) are mutated either (31, 31′) at the respective mutation site (or sites) of one parent library (11) or (31″) at the respective mutation site (or sites) of the other parent library (12). If the combined library (19) had been made up of more than two parent libraries, then probe nucleic acids mutated at the respective mutation sites of the further parent library (or libraries) could be encountered.

Again, the amount of probe nucleic acids (31, 31′, 31″) is adjusted as in example 1 to achieve a 5-fold oversampling in a later sequencing step.

FIG. 2B further extends FIG. 2A and shows that corresponding probe nucleic acids (32, 32′, 32′″, 33, 33′, 33″) are obtained from further combined libraries (created as libraries 19′ and 19″ according to FIG. 2A). The probe nucleic acid adjustment step is only shown for combined library 19 in FIG. 2A and is not shown in FIG. 2B to keep the figure simple; the adjustment is performed for all combined libraries to achieve a 5-fold oversampling.

After adjustment (not shown in FIG. 2B) of the amounts of probe nucleic acids, one probe nucleic acid (31) of each combined library (19) is combined with one probe nucleic acid (32, 33) of each other combined library (19′, 19″) respectively to obtain a mixture of probe nucleic acids (39). This combination is then repeated such that one probe nucleic acid (31′, 31″) is combined with another one probe nucleic acid (32′, 33′; 32″, 33″) of the further combined libraries (19′, 19″) to create further mixtures (39′, 39′) of probe nucleic acids. Thus, FIG. 2B shows a total of three repetitions of steps ii) and iii) according to the above description of the invention. An individual nucleic acid tag (not shown) is then linked to each mixed probe nucleic acids (39, 39′, 39″), for example by ligation or by chemical coupling. Thus, all probe nucleic acids of a first mixture 39 are affixed a first nucleic acid tag, the probe nucleic acids of a second mixture 39′ are affixed with a second nucleic acid tag and so on.

The specifically tagged mixed probe nucleic acids (39, 39′, 39″) are then mixed to obtain one mixture of tagged probe nucleic acids (40). The mixed and tagged probe nucleic acids (40) are then sequenced (not shown) as described in example 1 and a total list of sequences is obtained. The sequences are deconvoluted in a two-step process: In a first step the sequences are sorted according to the individual nucleic acid tag attached to the probe nucleic acid sequence. As the individual nucleic acid sequence tag is specific for each round of repetition of steps ii) and iii) as described above, it is possible to identify the respective probe nucleic acid mixture (39, 39′, 39″). And from the location of the mutation site or mutation sites of the respective probe nucleic acid sequence it is possible to determine, in a second deconvolution step, the parent library (11, 12) and corresponding member responsible for the respective probe nucleic acid (31, 31′, 31″; 32, 32′, 32″; 33, 33′, 33″).

1. Method for analysing a target nucleic acid, comprising the steps of
i) providing isolated members of a set of two or more site mutagenesis libraries, wherein the members of each site mutagenesis library comprise a target nucleic acid mutated at one or more library-specific mutation sites, and wherein the mutation sites of the two or more site mutagenesis libraries differ from each other,
ii) selecting one member of each site mutagenesis library,
iii) obtaining, for each member, a probe nucleic acid of the respectively mutated target nucleic acid comprising at least the nucleotides of the respective mutation sites and adjacent nucleotides for identification of the respective member,
iv) mixing the probe nucleic acids into one mixture, and
v) sequencing the probe nucleic acids of the mixture obtained in step iv) in parallel.

2. Method according to claim 1, wherein
steps ii) and iii) are repeated to select at least one additional member of one or more site mutagenesis libraries and
for each round of repetition of steps ii) and iii), the probe nucleic acids are marked by a round specific nucleic acid tag before step iv).

3. Method according to claim 1, wherein for at least one member selected in step ii) a property dependent on the target nucleic acid other than its sequence is determined.

4. Method according to claim 3, wherein the property is a property of the target nucleic acid or of a protein encoded by the target nucleic acid, and the property is selected from one or more of
expression,
secondary, tertiary or quarternary structure, folding efficiency, aggregation, multimerization
temperature stability, pH stability, solvent stability, detergent stability, protease stability, binding stability, solubility, protease activity, storage stability, storage stability in detergent, residual activity after storage, stability against proteolytic degradation,
changes of kinetic parameters, enzymatic activity at low temperatures.
change of substrate, substrate specificity, enantioselectivity, cofactor dependence, inhibitor specificity, inhibition kinetics,
immunogenicity, toxicity, allergenicity, wash performance,
location in the cytoplasm, spore, cell membrane, cell organelle lumen or membrane, cell wall, excretion.

5. Method according to claim 1,
(a) wherein each member comprises
only one mutation site, or
two or more non-adjacent mutation sites,
the mutation site or sites consisting of one or more adjacent nucleotides, and
(b) wherein the mutation sites of two or more libraries
partially overlap or
do not overlap.

6. Method according to claim 1, wherein the nucleotides of the mutation sites of one, two or more libraries
(a) differ from the target nucleic acid sequence by one or more arbitrary nucleotides and/or
(b) differ from the target nucleic acid sequence by one or more degenerate nucleotides independently selected from [AT], [CG], [AC], [GT], [AG], [CT], [CGT], [AGT], [ACT], or [ACG], or
(c) are selected from a predefined set of nucleotide or nucleotide sequences.

7. Site saturation mutagenesis method, comprising the steps of
i) creating, for each mutation site of a target nucleic acid, a library of mutants, wherein the mutants comprise mutations of the target nucleic acid only at the mutation site or sites specific for the respective library, and
ii) analysing the libraries by a method according to claim 1.

8. Sequence analyser, comprising
a) a constraint database containing definitions of mutation sites allowed in a mutant of a target nucleic acid, and
b) a sequencer for sequencing nucleic acids in parallel, and
c) a watchdog program which, when the sequencer is used for sequencing of nucleic acids allegedly conforming to the constraint definitions, indicates erroneous sequences not conforming with the constraint definitions of the constraint database or suppresses the output of erroneous sequences.

9. Sequence analyser, comprising
a) a definition database of library members, wherein the library members are mutants of a target nucleic acid and the position of mutations of the target nucleic acid is specific for the respective library,
b) a sequencer for sequencing nucleic acids in parallel, and
c) a deconvolution program for identification of library members according to the library member definition based on the sequences obtained by the sequencer, optionally wherein the sequence analyser is a sequence analyser according to claim 8.

10. Sequence analyser according to claim 9, further comprising
a property database comprising for at least one library member a quality of a property dependent on the target nucleic acid other than its sequence, and
a correlation program for creating a pairwise assignment of library member sequence to property quality of the respective library member.

11. Sequence analyser computer program, wherein the program is
a watchdog program for a sequence analyser according to claim 7, and/or
a deconvolution program for a sequence analyser and/or
a correlation program for a sequence analyser.
